(19) United States
(12) Patent Application Publication
(10) Pub. No.: US 2020/0206349 A1
(43) Pub. Date: Jul. 2, 2020

(54) Immunothermosensitive Composite, Kit For Treating Cancer, And Use Thereof

(71) Applicant: National Tsing Hua University, Hsinchu (TW)

(72) Inventors: Hsing-Wen Sung, Hsinchu City (TW); Po-Ming Chen, New Taipei City (TW); Wen-Yu Pan, Taipei City (TW); Cheng-Yu Wu, Changhua City (TW); Ching-Yen Yeh, Taipei City (TW); Po-Kai Luo, Minxiong Township (TW); Chun-Ju Chou, Taipei City (TW); Yung-Seng Pang, New Taipei City (TW); Yu-Miao Liu, Gongguan Township (TW)

(21) Appl. No.: 16/407,912

(22) Filed: May 9, 2019

(30) Foreign Application Priority Data
Dec. 26, 2018 (TW) 107147252

(51) Int. Cl. A61K 41/00; A61K 9/50; A61K 39/39; A61P 35/00; A61N 5/06

(52) U.S. Cl. CPC A61K 41/0028; A61K 9/5036; A61K 9/5031; A61K 9/5089; A61N 2005/067; A61P 35/00; A61N 5/062; A61N 5/0625; A61N 2005/0659; A61K 39/39

(57) ABSTRACT

An immunothermosensitive composition is provided in the present disclosure. The immunothermosensitive composition includes a carrier and an immune adjuvant. The shell is carrier formed by self-assembly of a hydrophilic amine-containing polymer and a conductive polymer to form a hydrophilic region and a hydrophobic region, and the hydrophilic region is located outside the hydrophobic region. The immune adjuvant is coated in the hydrophobic region of the carrier, wherein the immune adjuvant specifically binds to Toll-Like Receptor 7 (TLR7) and/or Toll-Like Receptor 8 (TLR8). The immunothermosensitive composition can absorb light energy to generate thermal energy and is maintained at a temperature greater than or equal to 39° C. and less than or equal to 45° C.

### RELATED APPLICATIONS

This application claims priority to Taiwan Application Serial Number 107147252, filed Dec. 26, 2018, which is herein incorporated by reference.

### BACKGROUND

### Technical Field

The present disclosure relates to a composition, a use thereof, and a kit including the composition. More particularly, the present disclosure relates to a pharmaceutical composition characterized by special physical form, a use thereof, and a kit including the pharmaceutical composition.

### Description of Related Art

Cancer, also known as malignancy, is a state of abnormal proliferation of cells, and these proliferating cells may invade other parts of the body as a disease caused by a malfunction in the control of cell division and proliferation. The number of people suffering from cancer worldwide has a growing trend. Cancer is one of the top ten causes of death and has been the top ten causes of death for twenty-seven consecutive years.

Conventional cancer treatments include surgery, radiation therapy, and pharmacotherapy (including chemotherapy, targeted therapy and currently popular immunotherapy). However, these methods have their shortcomings. Clinically, surgery can not completely remove tumor cells in most cases, which may cause tumor recurrence in patients. Furthermore, chemotherapy and radiation therapy often have extremely serious side effects for normal tissues. Therefore, hyperthermia therapy has gradually become mainstream.

The hyperthermia therapy not only can be used for killing tumor cells but also can affect the immune response of human body. In order to eradicate tumor cells, tumor cells are generally killed by local heating during hyperthermia therapy, and the heating temperature is usually greater than 55° C. Although the tumor cells can be permanently killed at such a high temperature, immune cells (such as antigen presenting cells or T cells) are also eliminated by high temperature penetrated into the microenvironment of the tumor cells. It is also inevitable that high temperature will cause thermal diffusion, causing damage to healthy tissue adjacent to the tumor cells and causing substantial discomfort to the patient. Therefore, the aforementioned problem needs to be solved.

### SUMMARY

According to one aspect of the present disclosure, an immunothermosensitive composition is provided. The immunothermosensitive composition includes a carrier and an immune adjuvant. The carrier is formed by self-assembly of a hydrophilic amine-containing polymer and a conductive polymer to form a hydrophilic region and a hydrophobic region, wherein the hydrophilic region is located outside the hydrophobic region, and the conductive polymer has a covalent π bond. The immune adjuvant is coated in the hydrophobic region of the carrier, wherein the immune adjuvant specifically binds to Toll-Like Receptor 7 (TLR7) and/or Toll-Like Receptor 8 (TLR8). The immunothermosensitive composition can absorb light energy to generate thermal energy and maintain a temperature greater than or equal to 39° C. and less than or equal to 45° C.

According to another aspect of the present disclosure, a kit for treating cancer is provided. The kit for treating cancer includes the immunothermosensitive composition according to the aforementioned aspect and a light supply device for irradiating the immunothermosensitive composition.

According to yet another aspect of the present disclosure, a method for treating cancer is provided. The method for treating cancer includes administering an effective amount of the immunothermosensitive composition according to the aforementioned aspect to a subject in need for a treatment of cancer.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiments, with reference made to the accompanying drawings as follows:

FIG. 1 is a structural schematic view showing an immunothermosensitive composition according to the present disclosure.

FIG. 2A is a transmission electron microscope image of a carrier according to one embodiment of the present disclosure.

FIG. 2B is a transmission electron microscope image of an immunothermosensitive composition according to one embodiment of the present disclosure.

FIG. 3A is an optical absorbance spectrum of an immunothermosensitive composition according to one embodiment of the present disclosure.

FIG. 3B is a temperature evolution curve of an immunothermosensitive composition after 808 nm NIR laser irradiation according to one embodiment of the present disclosure.

FIGS. 4A, 4B and 4C show effects of free R848, a carrier according to one embodiment of the present disclosure and an immunothermosensitive composition according to one embodiment of the present disclosure on cell viability of CT26 cells.

FIG. 5 shows analysis result of heat shock protein 70 expression of the CT26 cells after hyperthermia therapy.

FIGS. 6A and 6B show analysis results of cell uptake of the immunothermosensitive composition of the present disclosure by dendritic cells.

FIGS. 7A, 7B, 7C and 7D show analysis results of mature dendritic cell markers expression of dendritic cells treated with the immunothermosensitive composition of the present disclosure.

FIG. 8 shows analysis result of proinflammatory cytokines secreted by dendritic cells treated with the immunothermosensitive composition of the present disclosure.

FIGS. 9A and 9B show analysis results of photothermal effects of the immunothermosensitive composition of the present disclosure in experiment animals.

FIG. 10 is a schematic view showing a treatment strategy of the immunothermosensitive composition and a kit for treating cancer of the present disclosure in an animal treatment test.

FIG. 11A is a graph showing a size of each primary tumor of the Balb/c tumor mice after a treatment.

FIG. 11B is a survival curve of the Balb/c tumor mice after the treatment.

FIG. 12 shows analysis result T cell infiltration in the local tumor microenvironment of the Balb/c tumor mice after the treatment.

FIG. 13 is a graph showing a size of each rechallenged tumor of the Balb/c tumor mice after the treatment.

FIGS. 14A and 14B show enzyme-linked immunospot (ELISPOT) assay result of the Balb/c tumor mice after the treatment.

### DETAILED DESCRIPTION

The following descriptions of particular embodiments and examples are provided by way of illustration and not by way of limitation. Those skilled in the art will readily recognize a variety of noncritical parameters that could be changed or modified to yield essentially similar results.

Unless otherwise stated, the meanings of the scientific and technical terms used in the specification are the same as those of ordinary skill in the art. Furthermore, the nouns used in this specification are intended to cover the singular and plural terms of the term unless otherwise specified.

The term “individual” or “patient” refers to an animal that is capable of administering an immunothermosensitive composition and/or a kit for treating cancer of the present disclosure. Preferably, the animal is a mammal.

The term “cancer” refers to a non-solid tumor or a solid tumor. For example, cancer includes, but is not limited to, blood cancer, lymphoma, diaphyseal osteosarcoma, multiple myeloma, testicular cancer, thyroid cancer, prostate cancer, laryngeal cancer, cervical cancer, nasopharyngeal cancer, breast cancer, colorectal cancer, pancreatic cancer, head and neck cancer, esophageal cancer, rectal cancer, lung cancer, liver cancer, brain cancer, melanoma or skin cancer.

The term “about” means that the actual value falls within the acceptable standard error of the average, as determined by person having ordinary skill in the art. The scope, number, numerical values, and percentages used herein are modified by the term “about” unless example or otherwise stated. Therefore, unless otherwise indicated, the numerical values or parameters disclosed in the specification and the claims are approximate values and can be adjusted according to requirements.

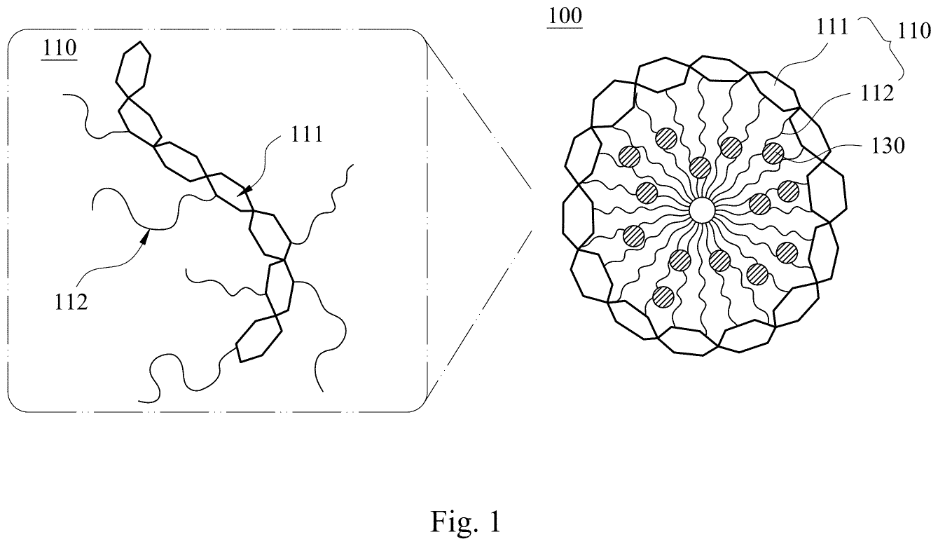

Please refer to FIG. 1, which is a structural schematic view showing an immunothermosensitive composition 100 according to the present disclosure. As shown in FIG. 1, the immunothermosensitive composition 100 can be a nanosphere, and includes a carrier 110 and an immune adjuvant 130. The carrier 110 is by self-assembly of a hydrophilic amine-containing polymer 111 and a conductive polymer 112 to form a hydrophilic region (not shown) and a hydrophobic region (not shown), and the hydrophilic region is located outside the hydrophobic region. The conductive polymer has a covalent π bond and has light absorbing characteristics, such as the light absorbing characteristic of ultraviolet light (UV), near infrared light (NIR), and far infrared light (FAR) or visible light (VIS), to convert the absorbed light energy into heat energy through the photothermal effect. The immune adjuvant 130 is coated in the hydrophobic region of the carrier 110, wherein the immune adjuvant 130 specifically binds to Toll-Like Receptor 7 (TLR7) and/or Toll-Like Receptor 8 (TLR8).

The hydrophilic amine-containing polymer 111 can be glycol chitosan (GCS), gelatin, O-carboxymethyl chitosan (CMOS), albumin, poly-L-lysine (PLL) or polyetherimide (PEI).

The conductive polymer 112 can be polyaniline (PANI), trans-polyacetylene (trans-PA), poly-p-phenylene (PPP), poly(p-phenylene vinylene) (PPV), poly(p-phenylene sulfide) (PPS), polypyrrole (PPy), polythiophene (PTh), poly(3,4-ethylenedioxythiophene) (PEDOT) or poly(9,9-di-n-octyl-2,7-fluorene) (PFO). The structural formula of the aforementioned conductive polymer 112 is shown in Table 1 below.

TABLE 1

| conductive | |
|---|---|
| polymer | structural formula |
| PANI | 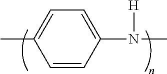 |
| trans-PA | 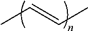 |
| PPP | 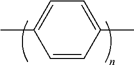 |
| PPV | 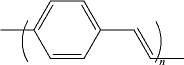 |
| PPS | 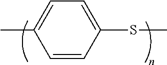 |
| PPy | 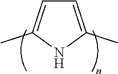 |
| PTh | 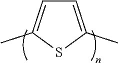 |
| PEDOT | 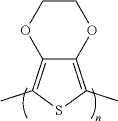 |
| PFO | 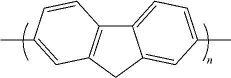 |

The immune adjuvant 130 can be an imidazoquinoline compound, a thiazoloquinoline compound or a benzoazepine analog. The imidazoquinoline compound can be resiquimod (R848), imiquimod (R837), gardiquimod, CL097 or 3M-003. The thiazoloquinoline compound can be CL075(3M-002). The benzoazepine analog can be TL8-506 or motolimod. The structural formula of the aforementioned immune adjuvant 130 is shown in Table 2 below.

TABLE 2

| immune | |
|---|---|
| adjuvant | structural formula |
| resiquimod (R848) | 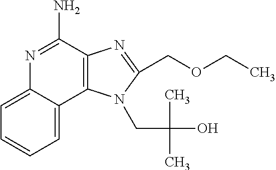 [SMILES: CCOCc1nc2c(N)nc3ccccc3c2n1CC(C)(C)O] |
| imiquimod (R837) | 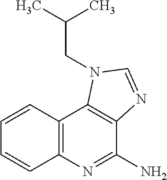 [SMILES: CC(C)Cn1cnc2c(N)nc3ccccc3c21] |
| gardiquimod | 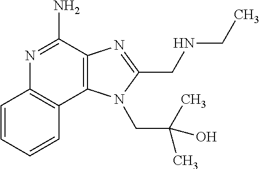 [SMILES: CCNCc1nc2c(N)nc3ccccc3c2n1CC(C)(C)O] |
| CL097 | 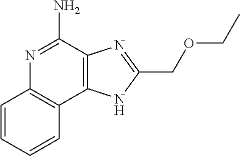 [SMILES: CCOCc1nc2c(N)nc3ccccc3c2[nH]1] |
| 3M-003 | 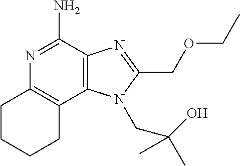 [SMILES: CCOCc1nc2c(N)nc3c(c2n1CC(C)(C)O)CCCC3] |
| CL075 (3M-002) | 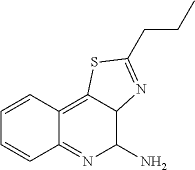 [SMILES: CCCC1=NC2C(=c3ccccc3=NC2N)S1] |
| TL8-506 | 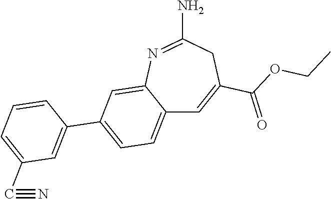 [SMILES: CCOC(=O)C1=Cc2ccc(-c3cccc(C#N)c3)cc2N=C(N)C1] |
| motolimod | 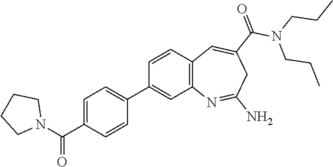 [SMILES: CCCN(CCC)C(=O)C1=Cc2ccc(-c3ccc(C(=O)N4CCCC4)cc3)cc2N=C(N)C1] |

Therefore, the immunothermosensitive composition 100 of the present disclosure can absorb light energy to generate thermal energy and maintain a temperature greater than or equal to 39° C. and less than or equal to 45° C. The thermal energy generated and maintained by the immunothermosensitive composition 100 of the present disclosure is mild that allows tumor cells to release tumor antigens. In addition, the immune adjuvant 130 can specifically bind to TLR 7 and/or TLR 8 to promote the initiation of an immune mechanism, activate a tumor-specific T cell response to recognize a tumor antigen, and convert the whole tumor into an in situ individualized vaccine that can inhibit the growth of the original tumor and develop effective anti-tumor immunity.

The aforementioned immunothermosensitive composition can be used as a pharmaceutical composition for treating cancer. For example, a pharmaceutical composition inhibiting cancer cell proliferation, a pharmaceutical composition for inhibiting cancer metastasis, a pharmaceutical composition for inhibiting cancer recurrence, or a pharmaceutical composition for triggering a tumor immune response. Preferably, the pharmaceutical composition can be a tumor vaccine.

The aforementioned immunothermosensitive composition can be cooperated with a light supply device as the kit for treating cancer. A light source of the light supply device can be ultraviolet light (UV), near infrared light (NIR), far infrared light (FIR) or visible light (VIS). The immunothermosensitive composition can absorb light energy generated by the light supply device to generate a mild thermal energy and maintain the mild thermal energy. Therefore, the immunothermosensitive composition has the dual effects of hyperthermia therapy and immunotherapy, thereby producing synergistic therapeutic effects, and greatly improving the cancer treatment effect.

The immunothermosensitive composition, the use thereof and the kit for treating cancer has been described as mentioned above. In the following, reference will now be made in detail to the present embodiments of the present disclosure, experiments and examples of which are illustrated in the accompanying drawings. The accompanied effects of the immunothermosensitive composition and the kit for treating cancer disclosed in the experiments and the examples for demonstrating the effect and the mechanism of the immunothermosensitive composition and the kit for treating cancer in the immunotherapy. However, the present disclosure is not limited thereto.

### EXPERIMENTS AND EXAMPLES

### I. The Immunothermosensitive Composition of the Present Disclosure and the Preparation Method Thereof

1.1 Preparation of the Immunothermosensitive Composition

To test the optimal preparation condition of the immunothermosensitive composition, the carrier without coated immune adjuvant is prepared in this experiment first. The hydrophilic amine-containing polymer used in this example is glycol chitosan (hereafter “GCS”), and the conductive polymer used in this example is polyaniline (hereafter “PANI”). PANI is grafted onto GCS to form PANI-GCS by the oxidative polymerization of aniline in the presence of GCS and ammonium persulfate (APS), a strong oxidizing agent. Briefly, an aqueous solution of GCS is firstly prepared by dissolving 100 mg GCS in 40 mL of 0.1 M HCl. Following the addition of 0.5 mM aniline to the GCS solution, an equimolar amount of APS is introduced to initiate polymerization at 4° C. Four hours later, the reaction solution is centrifuged at 5000 g for 15 minutes three times to remove the insoluble free PANI. The synthesized PANI-GCS, which is in the supernatant, is purified by dialysis using a dialysis bag (MWCO=12-14 kDa) against deionized (DI) water for two days. Finally, the resulting solution is lyophilized to yield the carrier PANI-GCS, wherein 1 mg carrier PANI-GCS contains approximately 30 μg PANI.

Further, the aforementioned prepared carrier PANI-GCS is coated with the immune adjuvant by a cosolvent evaporation method to prepare the immunothermosensitive composition of the present disclosure, and then purified by a dialysis method. The immune adjuvant used in this example is resiquimod (hereafter “R848”). Briefly, 2 mg PANI-GCS is dissolved in 1 ml water and mixed with 1 ml methanol. R848 powder is first dissolved in DMSO (10 mg/ml) as then predetermined amount of R848 solution (0 μL to 40 μL) are mixed with 1 ml of 1:1 (v/v) water/methanol and added to the PANI-GCS solution. The solution is mixing by vortex then transferred into a round bottom flask, the organic solvent is removed under reduced pressure at 25° C. for 10 minutes by a rotavapor. The remaining solution is then transferred into a dialysis tubing (molecular weight cutoff=12,000-14,000) to remove the residual solvents and free R848 against deionized water for 2 days. Finally, the resulting solution is lyophilized to obtain the immunothermosensitive composition PANI-GCS-R848. The amount of free R848 remaining in the dialysis water are determined by reverse-phase high-performance liquid chromatography (HPLC). The drug loading content (LC) and drug loading efficiency (LE) of the immunothermosensitive composition PANI-GCS-R848 are calculated using the equations listed below:

$$\begin{matrix}LC ( \% ) = \frac{Total amount of R 848 added - Free R 848}{Weight of immunothermosensitive composition} \times 100 \% ; & equation I \\ LE ( \% ) = \frac{Total amount of R 848 added - Free R 848}{Total amount of R 848 added} \times 100 \% . & equation II\end{matrix}$$

### 1.2 Structure and Characterization Analysis of the Immunothermosensitive Composition

Please refer to Table 3, which shows the drug loading content (LC) and drug loading efficiency (LE) of the immunothermosensitive composition PANI-GCS-R848 prepared by different weight ratios of R848 (immune adjuvant) to PANI-GCS (carrier).

TABLE 3

| weight ratio of R848 to | | |
|---|---|---|
| PANI-GCS | LC (%) | LE (%) |
| 5%:95% | 3.7 ± 0.7 | 77.7 ± 16.0 |
| 10%:90% | 7.0 ± 1.1 | 75.8 ± 13.0 |
| 15%:85% | 9.1 ± 1.0 | 66.6 ± 8.1 |
| 20%:80% | 9.4 ± 1.5 | 52.3 ± 9.3 |
| 25%:75% | N/A | N/A |

In Table 3, when the weight ratio of R848 to PANI-GCS is 5%:95%, the drug loading content is 3.7±0.7%, and the drug loading efficiency is 77.7±16.0%; when the weight ratio of R848 to PANI-GCS is 10%:90%, the drug loading content is 7.0±1.1%, the drug loading efficiency is 75.8±13.0%; when the weight ratio of R848 to PANI-GCS is 15%:85%, the drug loading content is 9.1±1.0%, and the drug loading efficiency is 66.6±8.1%; when the weight ratio of R848 to PANI-GCS is 20%:80%, the drug loading content is 9.4±1.5%, and the drug loading efficiency is 52.3±9.3%. The results indicate that the immunothermosensitive composition PANI-GCS-R848 of the present disclosure has a good loading effect, and the drug loading content of the immunothermosensitive composition PANI-GCS-R848 is increased with an increase in the feeding ratio of R848. The drug loading content of the immunothermosensitive composition PANI-GCS-R848 is maximized at the R848 to PANI-GCS weight ratio of 20%:80%. The drug loading efficiency of the immunothermosensitive composition PANI-GCS-R848 is decreased with an increase in the feeding ratio of R848. When the weight ratio of R848 to PANI-GCS is 25%:75%, the drug loading content and the drug loading efficiency of the immunothermosensitive composition PANI-GCS-R848 cannot be measured due to the instability of the immunothermosensitive composition PANI-GCS-R848.

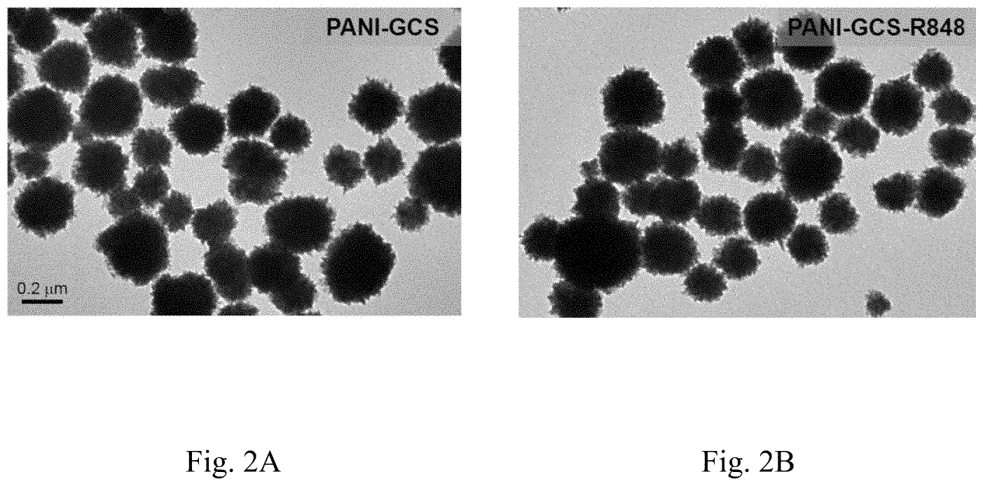

The carrier PANI-GCS and the immunothermosensitive composition PANI-GCS-R848 will self-assemble due to their hydrophilicity and hydrophobicity. The surface charge of the carrier PANI-GCS and the immunothermosensitive composition PANI-GCS-R848 that are self-assembled in phosphate-buffered saline (PBS) is measured by dynamic light scattering using a Zetasizer (Zetasizer 3000HS; Malvern Instruments, Worcestershire, UK), while the morphology and size of the self-assembled carrier PANI-GCS and the self-assembled immunothermosensitive composition PANI-GCS-R848, after they had been stained with osmium tetroxide (OsO₄), are examined using transmission electron microscopy (TEM; JEM-2100F, JEOL Technics, Tokyo, Japan). Please refer to FIGS. 2A and 2B. FIG. 2A is a transmission electron microscope image of the carrier PANI-GCS according to one embodiment of the present disclosure. FIG. 2B is a transmission electron microscope image of the immunothermosensitive composition PANI-GCS-R848 according to one embodiment of the present disclosure. In FIG. 2A, the carrier PANI-GCS is predominantly spherical in shape with a mean size of 161.3±39.4 nm (n=6 batches). In FIG. 2B, the immunothermosensitive composition PANI-GCS-R848 is predominantly spherical in shape with a mean size of 167.9±44.1 nm (n=6 batches). In addition, as shown in FIGS. 2A and 2B, the carrier PANI-GCS and the immunothermosensitive composition PANI-GCS-R848 can be well dispersed in aqueous solutions. The carrier PANI-GCS has a zeta potential of 33.2±1.9 mV, and the immunothermosensitive composition PANI-GCS-R848 has a zeta potential of 31.9±3.2 mV.

To confirm that the immunothermosensitive composition of the present disclosure can absorb light energy to generate thermal energy and maintain the temperature greater than or equal to 39° C. and less than or equal to 45° C., the UV-vis-NIR optical properties of GCS, the carrier PANI-GCS, and the immunothermosensitive composition PANI-GCS-R848 in PBS are recorded using a SpectraMax M5 Microplate Reader (Molecular Devices, Sunnyvale, Calif., USA). To elucidate the photothermal ability of the carrier PANI-GCS and the immunothermosensitive composition PANI-GCS-R848, test samples are dispersed in PBS at 150 μg/ml in a 48-well plate, irradiated using an 808 nm NIR laser (Tanyu Tech., Kaohsiung, Taiwan), and then detected the temperature change pattern.

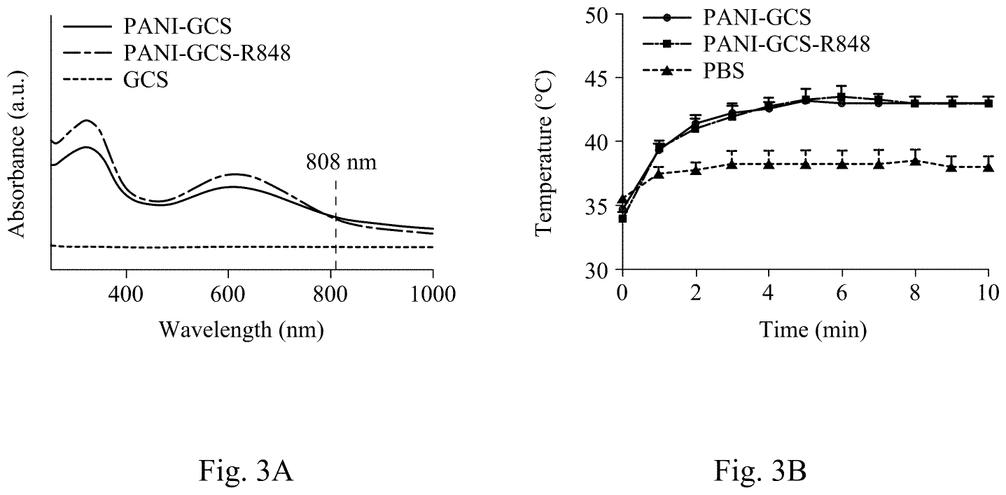

Please refer to FIGS. 3A and 3B. FIG. 3A is an optical absorbance spectrum of an immunothermosensitive composition according to one embodiment of the present disclosure. FIG. 3B is a temperature evolution curve of an immunothermosensitive composition after 808 nm NIR laser irradiation according to one embodiment of the present disclosure. In FIG. 3A, both the carrier PANI-GCS and the immunothermosensitive composition PANI-GCS-R848 exhibit a greater NIR light absorbance than GCS at 808 nm. In FIG. 3B, compared with the PBS group, the temperatures of the PBS containing the carrier PANI-GCS and the immunothermosensitive composition PANI-GCS-R848 can rapidly increase to 44° C. within 3 minutes of irradiating the 808 nm NIR laser, and can maintain the temperature at 44° C.

### II. Use of the Immunothermosensitive Composition of the Present Disclosure

### 2.1 Cytotoxic Effects of the Immunothermosensitive Composition of the Present Disclosure

To determine the safety and safe dose of the immunothermosensitive composition of the present disclosure to tumor cells, cell viability assay is performed on murine colon carcinoma cell line CT26 (hereinafter “CT26 cells”) with different doses of the immunothermosensitive composition PANI-GCS-R848.

The CT26 cells are maintained in Roswell Park Memorial Institute (RPMI)-1640 medium supplemented with 10% fetal bovine serum (FBS) at 37° C. in a 5% CO₂ humidified incubator.

The cytotoxicity of the immunothermosensitive composition PANI-GCS-R848 is examined by incubating the immunothermosensitive composition PANI-GCS-R848 at varying concentrations (6.25 μg/mL to 100 μg/mL) with the CT26 cells using a Cell Titer-Glo assay. The experiment further includes untreated CT26 cells, the CT26 cells treated with free R848 or the CT26 cells treated with the carrier PANI-GCS as a control. The concentration of the carrier PANI-GCS also ranges from 6.25 μg/mL to 100 μg/mL, and the concentration of the free R848 used in the experiment ranges from 0.625 μg/mL to 10 μg/mL in order to simulate the concentration of R848 loaded in the immunothermosensitive composition PANI-GCS-R848. The CT26 cells are seeded in 96-well plates at 1×10⁴ cells/well for 24 hours. Then the aforementioned different concentrations of immunothermosensitive composition PANI-GCS-R848, carrier PANI-GCS and R848 are incubated with the CT26 cells for following 24 hours at 37° C. The viability of the CT26 cells is evaluated by the CellTiter-Glo® Luminescent Cell Viability Assay kit (Promega, Madison, Wis., USA).

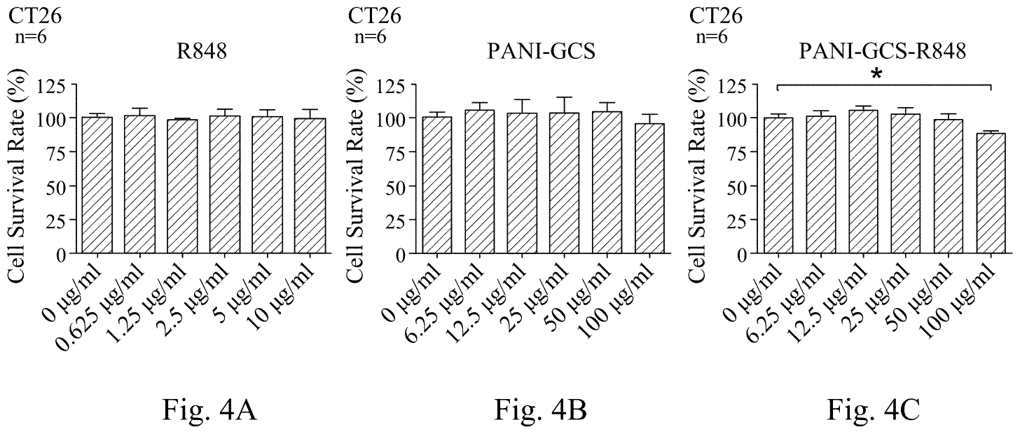

Please refer to FIGS. 4A, 4B and 4C, which show effects of free R848, the carrier PANI-GCS and the immunothermosensitive composition PANI-GCS-R848 on cell viability of the CT26 cells. FIG. 4A is a graph showing the cell viability result of the CT26 cells treated with free R848, FIG. 4B is a graph showing the cell viability result of the CT26 cells treated with the carrier PANI-GCS, and FIG. 4C is a graph showing the cell viability result of the CT26 cells treated with the immunothermosensitive composition PANI-GCS-R848, wherein results are represented as mean±SD, n=6, and * indicates p<0.05 compared to the untreated control. In FIGS. 4A to 4C, different concentrations of free R848, carrier PANI-GCS and the immunothermosensitive composition PANI-GCS-R848 are not significantly cytotoxic to the CT26 cells, indicating that the immunothermosensitive composition of the present disclosure has good biocompatibility and low toxicity characteristic.

### 2.2 Hyperthermia Therapy Induces Tumor Cell Damage and Releases Heat Shock Protein 70

Recent studies have reported that local hyperthermia therapy to the tumor cells at relative temperature to 41° C. to 45° C. can improve anti-tumor immunity by certain physiological effects, such as regulation of heat shock protein 70 (HSP70) on the tumor cell membrane.

To assess whether the hyperthermia therapy can induce tumor cell damage and release HSP70 from tumor cells, the CT26 cells are incubated with the carrier PANI-GCS and then mildly heated to 44° C. under NIR light irradiation for 10 minutes (hereafter “NIR-44° C.”). The experiment further includes the untreated CT26 cells (hereafter “37° C.”) or the CT26 cells treated with NIR light only (hereafter “NIR”) as a control. After different treatments, the CT26 cells of different groups are incubated at 37° C. for 24 hours. The expression of HSP70 is measured by Human/Mouse/Rat Total HSP70/HSPA1A DuoSet IC ELISA kit (R&D Systems, Minneapolis, Minn.).

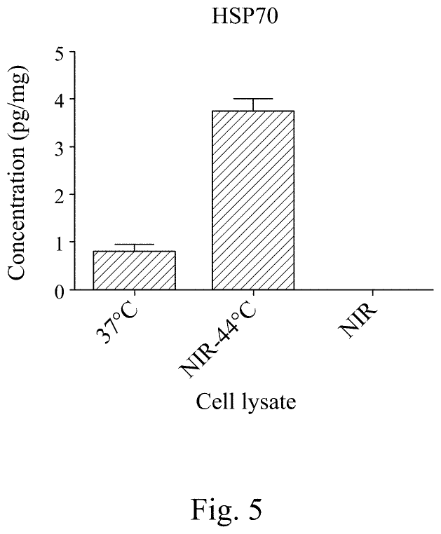

Please refer to FIG. 5, which shows analysis result of heat shock protein 70 expression of the CT26 cells after the hyperthermia therapy. In comparison to that of the untreated CT26 cells, the cellular expression level of HSP70 in the CT26 cells that are treated with the carrier PANI-GCS and the hyperthermia therapy by NIR light irradiation is significantly increased.

### 2.3 Cell Uptake of the Immunothermosensitive Composition of the Present Disclosure by Dendritic Cells and Activation of the Dendritic Cells

The dendritic cells used in the experiment are dendritic cells differentiated from bone marrow cells (BMDC). Briefly, the bone marrow cells are isolated from the femurs and tibias of Balb/c mice, and are cultured in RPMI-1640 medium supplemented with 10% heat inactivated FBS, 50 μM of 2-mercaptoethanol (Thermo Fisher Scientific, Waltham, Mass., USA), 1% penicillin/streptomycin and recombinant murine granulocyte-macrophage colony stimulating factor (GM-CSF, 10 ng/mL, PeproTech, Rocky Hill, N.J., USA). After 10 days, suspended immature BMDCs are collected for further experiments. To analyze the cellular internalization, BMDCs (1×10⁶ cells/mL) are incubated with 50 μg/mL Alexa Fluor@ 633 labeled immunothermosensitive composition PANI-GCS-R848 (hereafter “f-PANI-GCS-R848”) for 48 hours. The cellular uptake of the f-PANI-GCS-R848 by the BMDCs is measured by flow cytometry and immunofluorescence staining. The experiment further includes the BMDCs treated with medium only as a control. Flow cytometry is performed to measure the cell uptake of f-PANI-GCS-R848 by BD Accuri™ C6 flow cytometer (BD Biosciences, San Jose, Calif., USA), and data are analyzed using FlowJo (Treestar, Ashland, Oreg., USA). In immunofluorescence staining, LysoTracker is used as a cell marker for endosome. The BMDCs are incubated with LysoTracker™ Red DND-99 (Thermo Fischer Scientific) containing medium for 2 hours, washed, and stained with 4′,6-diamidino-2-phenylindole (DAPI) in PBS for 10 minutes. Finally, the BMDCs are observed and imaged under a confocal laser scanning microscopy (CLSM, LSM 780, Carl Zeiss, Jena, Germany).

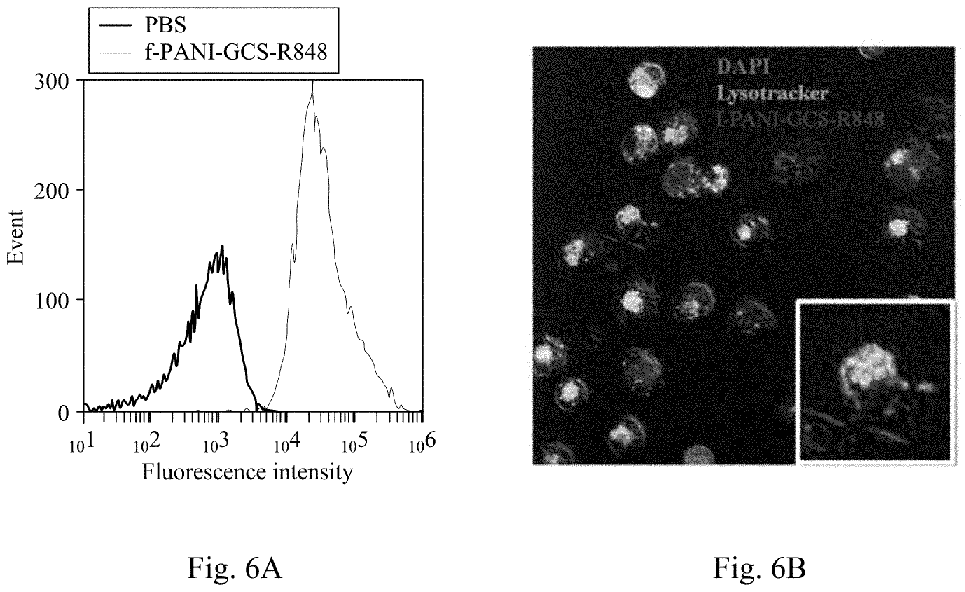

Please refer to FIGS. 6A and 6B, which show analysis results of cell uptake of the immunothermosensitive composition of the present disclosure by dendritic cells, wherein FIG. 6A shows the analysis result of flow cytometry, and FIG. 6B shows the analysis result of immunofluorescence staining. Fluorescence signal can be detected in the BMDCs co-cultured with f-PANI-GCS-R848. In FIG. 6B, the position of the fluorescent signal of f-PANI-GCS-R848 in BMDC overlaps with LysoTracker, indicating that f-PANI-GCS-R848 can indeed be taken into the BMDCs by phagocytosis.

To investigate the activation of BMDCs by the immunothermosensitive composition of the present disclosure, BMDCs are stimulated with 5 μg/mL of free R848, 50 μg/mL of the carrier PANI-GCS (represented as “PANI-GCS”), 50 μg/mL of the immunothermosensitive composition PANI-GCS-R848 (represented as “PANI-GCS-R848”) for 48 hours. The BMDCs and the culture supernatants are then collected separately. The expression of mature dendritic cell markers CD80 and CD86 in the treated BMDCs are analyzed by flow cytometry, and the concentrations of inflammatory cytokines secreted by mature dendritic cells in the supernatant are measured by magnetic beads arrays (CBA, BD Bioscience). The experiment further includes the BMDCs treated with medium only as negative controls (represented as “PBS”). The flow cytometry includes follow steps. The BMDCs are incubated with APC-conjugated anti-mouse CD80 antibody (eBioscience) or FITC-conjugated anti-mouse CD86 Antibody (eBioscience), and then analyzed by BD Accuri™ C6 flow cytometer.

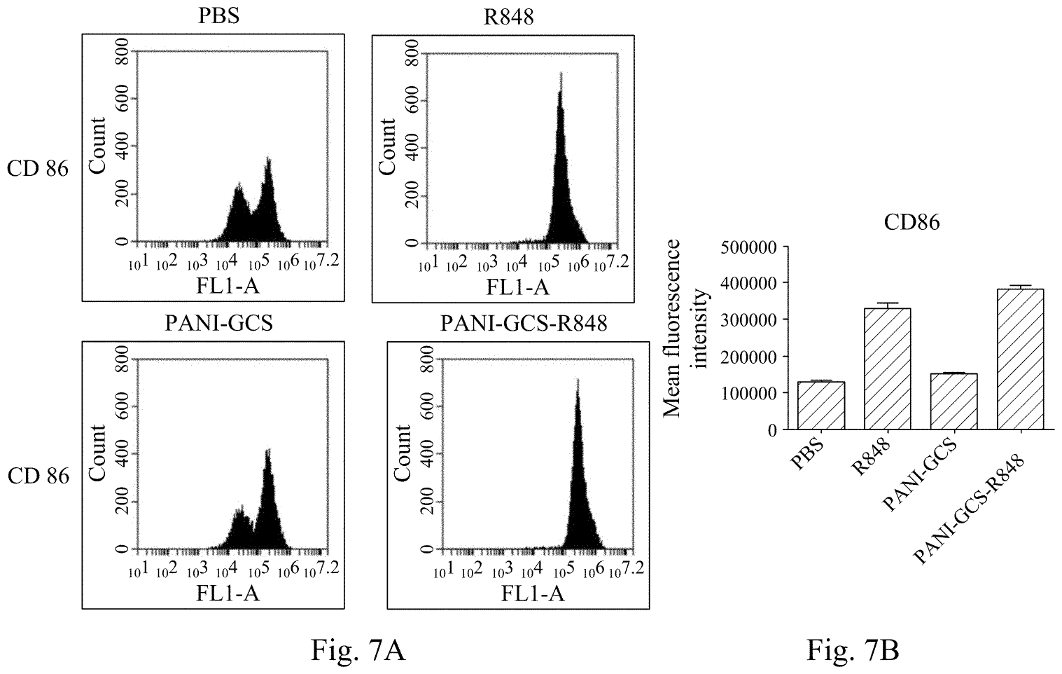

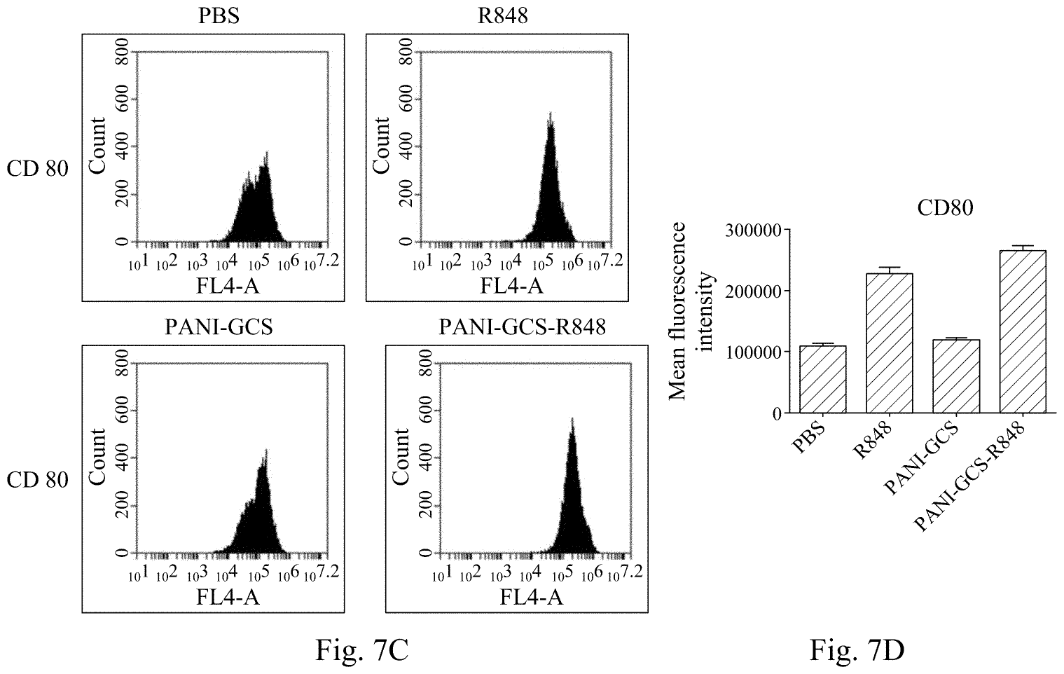

Please refer to FIGS. 7A, 7B, 7C and 7D, which show analysis results of mature dendritic cell markers expression of dendritic cells treated with the immunothermosensitive composition PANI-GCS-R848. FIGS. 7A and 7B show the analysis results of CD86 expression, and FIGS. 7C and 7D show the analysis results of CD80 expression. In FIGS. 7A to 7D, compared to the untreated BMDCs and the BMDCs treated with the carrier PANI-GCS, the expression levels of CD86 and CD80 on the BMDCs treated with the immunothermosensitive composition PANI-GCS-R848 are significantly elevated. The result indicates that the BMDCs treated with the immunothermosensitive composition PANI-GCS-R848 are indeed activated as mature dendritic cells.

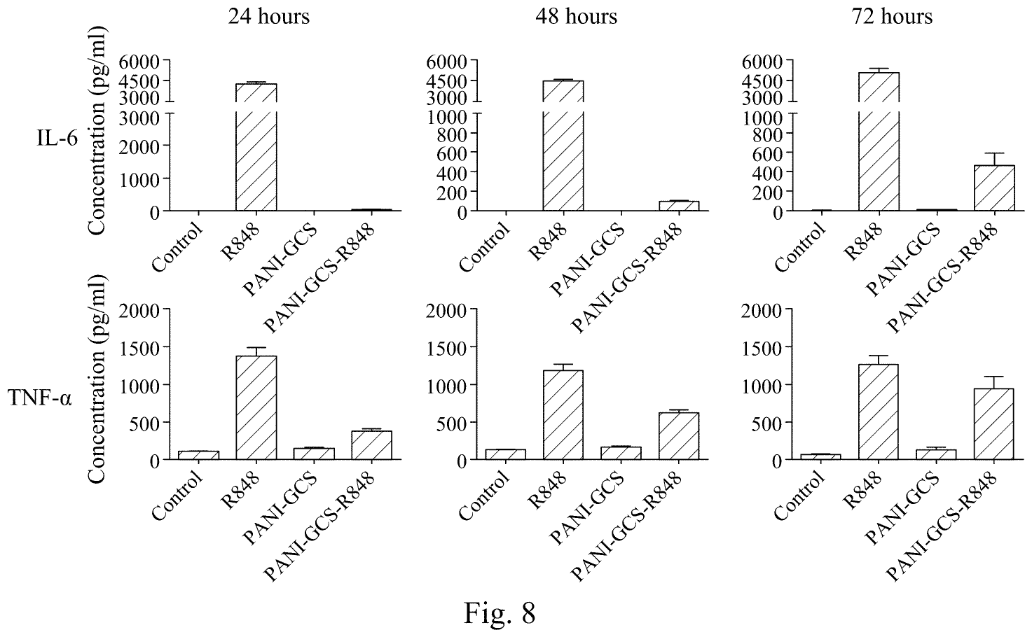

Please refer to FIG. 8, which shows analysis result of proinflammatory cytokines secreted by the BMDCs treated with the immunothermosensitive composition PANI-GCS-R848, wherein the proinflammatory cytokines analyzed are IL-6 and TNF-α. In FIG. 8, the BMDCs treated with free R848 can secrete notable levels of IL-6 and TNF-α to the culture medium by 24 hours, but the levels of IL-6 and TNF-α of the BMDCs treated with free R848 do not change significantly at 48 hours and 72 hours. Conversely, although the BMDCs treated with the immunothermosensitive composition PANI-GCS-R848 secrete markedly less IL-6 and TNF-α at 24 hours, their secretion levels of IL-6 and TNF-α are substantially upregulated afterwards. The results indicate that the immunothermosensitive composition PANI-GCS-R848 which encapsulates R848 in the carrier PANI-GCS can be gradually degraded and released in the intracellular body of the cell to achieve the effect of sustained release of R848.

### 2.4 Therapeutic Effect of the Immunothermosensitive Composition of the Present Disclosure for Treating Cancer

Experimental animals used in the experiments are Balb/c mice (6-8 weeks old), which are purchased from BioLASCO Taiwan Co., Ltd. (Taipei, Taiwan). All the animal experiments are performed according to “Guide for the Care and Use of Laboratory Animals” developed by the Institute of Laboratory Animal Resources, National Research Council. Balb/c mice are inoculated with 1×10⁶ CT26 cells subcutaneously on their right flanks (primary tumor). Fourteen days later when the tumor diameter is about 5 mm (or the tumor volumes reach 150-200 mm³), the Balb/c tumor mice are established and further used to test the cancer treatment effects on the immunothermosensitive composition and the kit for treating cancer of the present disclosure, and whether the immunothermosensitive composition and the kit for treating cancer of the present disclosure can achieve the dual effects of hyperthermia therapy and immunotherapy.

The photothermal ability of the immunothermosensitive composition of the present disclosure is evaluated in vivo in the Balb/c tumor mice. Following intratumoral injection of the immunothermosensitive composition PANI-GCS-R848 that are suspended in PBS to the Balb/c tumor mice, the tumors are individually exposed to the NIR laser at a power density of 0.9 W/cm² for 10 minutes (represented as “PANI-GCS-R848+NIR”). The local temperatures are recorded by an IR thermal camera. The experiment further includes the Balb/c tumor mice that received the carrier PANI-GCS (represented as “PANI-GCS+NIR”) or PBS (represented as “NIR”) alone served as controls.

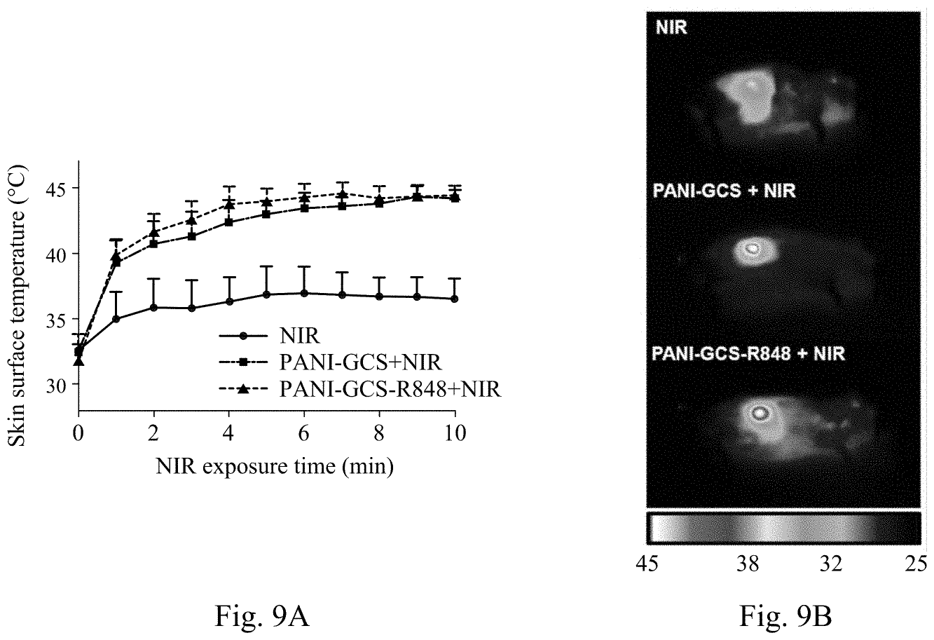

Please refer to FIGS. 9A and 9B, which show analysis results of photothermal effects of the immunothermosensitive composition of the present disclosure in experiment animals, wherein FIG. 9A shows quantitative temperature evolution curves, and FIG. 9B shows thermographic images. In FIGS. 9A and 9B, upon 808 nm NIR laser exposure, compared to the Balb/c tumor mice received PBS alone, the Balb/c tumor mice treated with the carrier PANI-GCS or the immunothermosensitive composition PANI-GCS-R848 exhibit a rapidly rise in temperature from 37° C. to 45° C. within 4 minutes, and maintain to the temperature range.

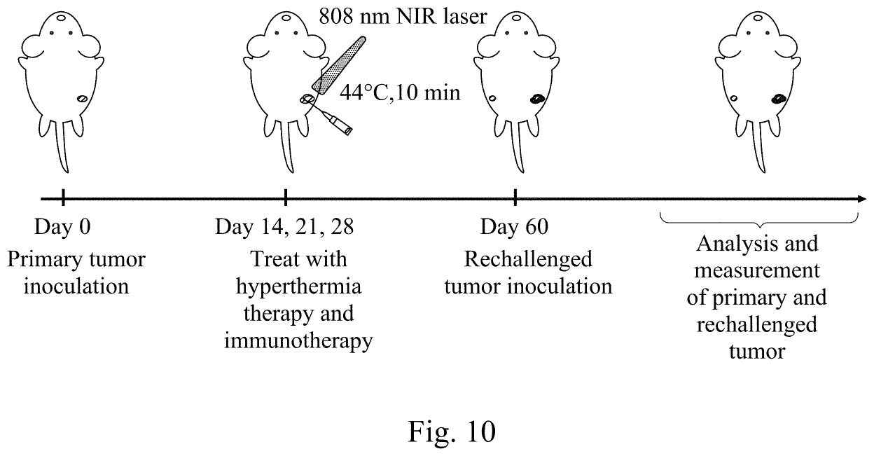

Further, the cancer treatment effects on the immunothermosensitive composition and the kit for treating cancer of the present disclosure is confirmed in the experiment. Please refer to FIG. 10, which is a schematic view showing a treatment strategy of the immunothermosensitive composition and the kit for treating cancer of the present disclosure in an animal treatment test. On day 0, Balb/c mice are inoculated with 1×10⁶ CT26 cells subcutaneously on their right flanks to produce a primary tumor. Fourteen days later when the tumor diameter is about 5 mm (or the tumor volumes reach 150-200 mm³), the Balb/c tumor mice are treated on day 14, day 21 and day 28. The treatment is that the Balb/c tumor mice are treated with the immunothermosensitive composition PANI-GCS-R848 and then the tumors of the Balb/c tumor mice are exposed to the 808 nm NIR laser at the power density of 0.9 W/cm² for 10 minutes (represented as “PANI-GCS-R848+NIR”). This process is repeated every seven days for a total of three treatment sessions. The size of each primary tumor, which is estimated as length×width×height×π/6, is assessed using a pair of caliper every 2-3 days. The Balb/c tumor mice are humanely sacrificed when the primary tumor reached a size of 3,000 mm³. If the primary tumor of the Balb/c tumor mouse has completely disappeared on day 60, the same Balb/c tumor mouse is inoculated with 1×10⁵ CT26 cells subcutaneously on its left flank to produce a rechallenged tumor. The Balb/c tumor mice with the rechallenged tumor are no longer treated, but the size of each rechallenged tumor is observed and recorded.

In addition, the treatment on day 14, day 21 and day 28 further includes six treatment controls, which are the Balb/c tumor mice treated with PBS only (represented as “PBS”), the Balb/c tumor mice treated with free R848 only (represented as “R848”), the Balb/c tumor mice treated with the carrier PANI-GCS only (represented as “PANI-GCS”), the Balb/c tumor mice treated with the immunothermosensitive composition PANI-GCS-R848 only (represented as “PANI-GCS-R848”), the Balb/c tumor mice treated with PBS and exposed to the 808 nm NIR laser (represented as “PBS+NIR”), and the Balb/c tumor mice treated with the carrier PANI-GCS and exposed to the 808 nm NIR laser (represented as “PANI-GCS+NIR”). The number of the Balb/c tumor mice in each group is 10-21.

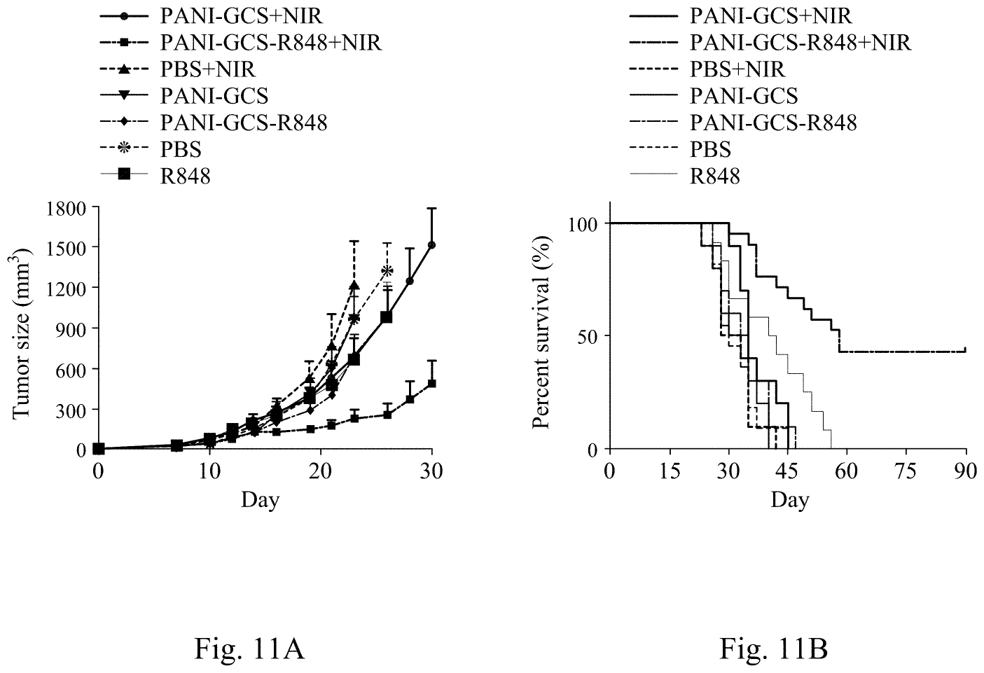

Please refer to FIG. 11A, which is a graph showing the size of each primary tumor of the Balb/c tumor mice after the treatment. In FIG. 11A, the growth of the tumor is slightly delayed in the Balb/c tumor mice treated with hyperthermia therapy only (PANI-GCS+NIR) or immunotherapy only (PANI-GCS-R848 or R848). In contrast, in the Balb/c tumor mice treated with the kit for treating cancer of the present disclosure (PANI-GCS-R848+NIR), the progression of the primary tumor can be effectively inhibited.

Please refer to FIG. 11B, which is a survival curve of the Balb/c tumor mice after the treatment. In FIG. 11B, 43% of the Balb/c tumor mice treated with the kit for treating cancer of the present disclosure (PANI-GCS-R848+NIR) still survived on day 90, and they are free of detectable primary tumors. By contract, the Balb/c tumor mice receiving other treatments died within 60 days due to excessive tumors.

It is speculated that the kit for treating cancer of the present disclosure achieves such excellent cancer treatment effects by modulating the tumor microenvironment. Therefore, the T cell infiltration in the local tumor microenvironment of the Balb/c tumor mice after treatment is further investigated in the experiment. On day 4 following the first cycle of various treatments, the Balb/c tumor mice are sacrificed, and sections of residual primary tumors are collected and analyzed by histological staining. Changes in primary tumors after treatment are observed by H&E staining, and In Situ Cell Death Detection Kit (TUNEL assay, Roche, Mannheim, Germany) is used to evaluate whether the treatment promotes apoptosis of tumor cells in the Balb/c tumor mice after treatment. Sections of residual primary tumors are stained with the fluorescent labeled anti-CD3 antibody and the fluorescent labeled anti-granzyme B antibody to label the CD3⁺ T cells in the tumor and the granzyme B secreted by the T cells, and then stained with DAPI to label the nucleus position. The infiltration of immune cells in the tissue is observed and imaged using Zeiss LSM 780.

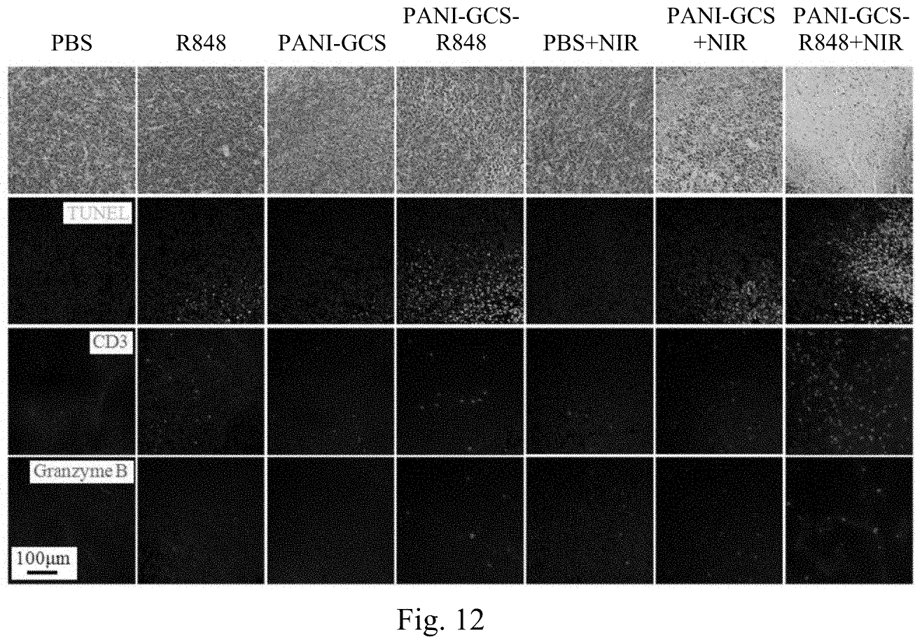

Please refer to FIG. 12, which shows analysis result T cell infiltration in the local tumor microenvironment of the Balb/c tumor mice after the treatment. In FIG. 12, the Balb/c tumor mice treated with the kit for treating cancer of the present disclosure (PANI-GCS-R848+NIR) can most effectively induce apoptosis, can attract the most CD3⁺ T cells, and can secrete most granzyme B, thus achieving such excellent cancer treatment effects.

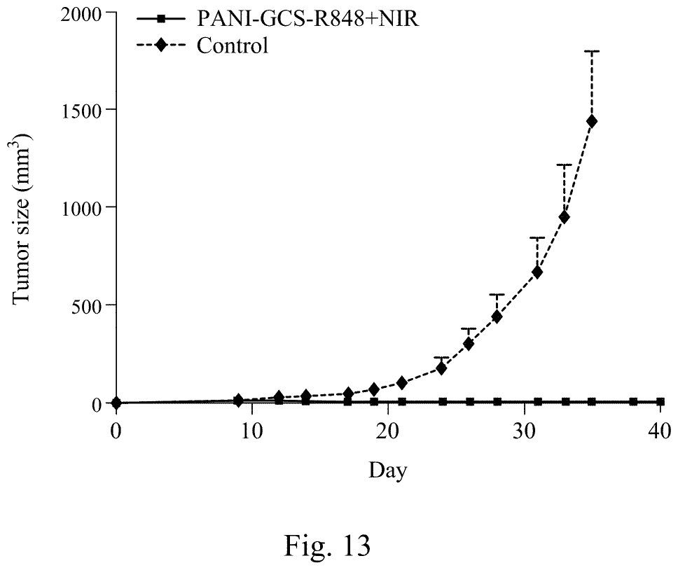

Please refer to FIG. 13, which is a graph showing a size of each rechallenged tumor of the Balb/c tumor mice after the treatment, wherein “PANI-GCS-R848+NIR” represents the Balb/c tumor mice in which the primary tumor has completely disappeared on day 60 and then are inoculated with the CT26 cells subcutaneously on the left flank after the treatment of the kit for treating cancer of the present disclosure. A new batch of Balb/c mice are inoculated with the CT26 cells subcutaneously on the left flank to produce a rechallenged tumor only as a control. In FIG. 13, in the Balb/c tumor mice treated with the kit for treating cancer of the present disclosure, the CT26 cells which are inoculated subcutaneously on the left flank do not grow into the rechallenged tumor at all. The result indicates that the kit for treating cancer of the present disclosure can induce an anti-tumor durable immunological memory in the Balb/c tumor mice against tumor metastasis or recurrence.

Further, whether the kit for treating cancer can induce the durable anti-tumor immunological memory in the Balb/c tumor mice is assessed ex vivo by an enzyme-linked immunospot (ELISPOT) assay in the experiment. After the tumor is treated with the kit for treating cancer of the present disclosure, a group of CD8⁺ T cells having memory for the tumor antigen exists in the spleen. Accordingly, when stimulated again by the tumor antigen, the CD8⁺ T cells having memory for the tumor antigen rapidly activate and secrete interferon-γ (IFN-γ), which can be detected by enzyme-linked immunospot assay. For the analysis of the ex vivo production of IFN-γ by CD8⁺ T cells, the spleens are harvested from the Balb/c tumor mice treated with kit for treating cancer of the present disclosure which had successfully rejected the tumor rechallenge. The spleen tissues are ground and filtered through a 40 μm Cell Strainer to remove the residue, and then the red blood cells are dissolved and removed using ACK Lysing Buffer. The isolated T cells are stimulated with the CT26-specific peptide- (AH1, SPSYVYHQF) pulsed syngeneic spleen cells. Twenty-four hours later, the productions of IFN-γ by the stimulated T cells are then assessed by ELISpot Mouse IFN-γ Kit (R&D Systems, Minneapolis, Minn.), and signals of the formed spots are evaluated by C.T.L. ImmunoSPOT Analyzer (Cellular Technology, OH, USA). The Balb/c mice at the same age without any treatment are used as controls in the experiment.

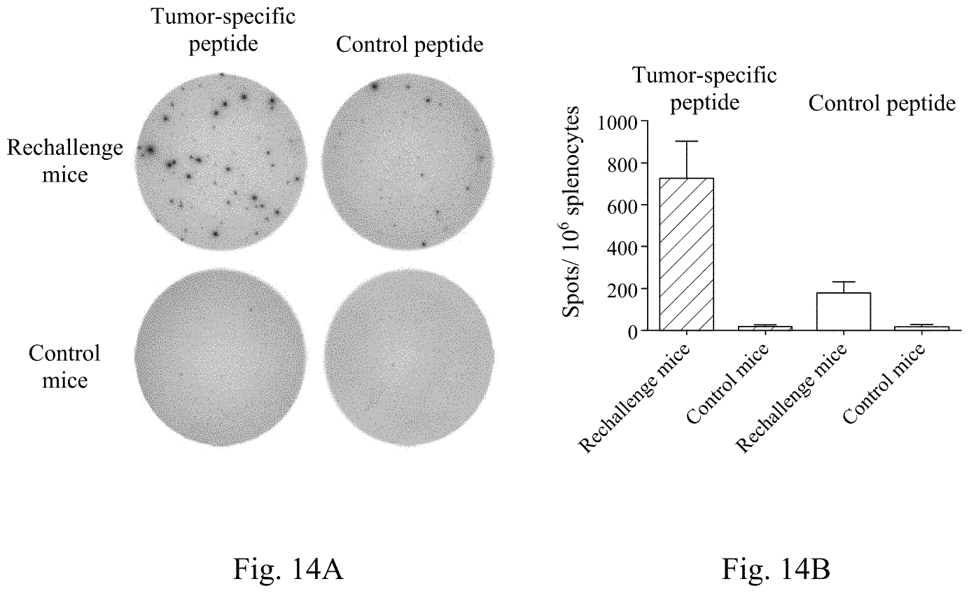

Please refer to FIGS. 14A and 14B, which show enzyme-linked immunospot assay result of the Balb/c tumor mice after the treatment, wherein FIG. 14B is a graph of quantitative result of FIG. 14A. In FIGS. 14A and 14B, the Balb/c tumor mice treated with the kit for treating cancer of the present disclosure have T cells that are specific to tumor-associated peptides. The result indicates that the kit for treating cancer of the present disclosure can induce the anti-tumor immunological memory in Balb/c tumor mice.

To sum up, the immunothermosensitive composition of the present disclosure can absorb light energy to generate thermal energy and maintain a temperature greater than or equal to 39° C. and less than or equal to 45° C., so that the immunothermosensitive composition of the present disclosure can induce tumor cells releasing heat shock proteins and tumor antigens, thereby increasing the recognition of immune antigenicity. The immune adjuvant coated in the immunothermosensitive composition can specifically bind to toll-like receptors and activate the dendritic cells to present the tumor antigen on the cell surface, thereby activating the tumor-specific T cell response and identifying the tumor antigen. Thus the immunothermosensitive composition of the present disclosure can trigger specific immune response to convert the whole tumor into the in situ individualized vaccine that can inhibit the growth of the original tumor and develop effective anti-tumor immunity. Therefore, the immunothermosensitive composition of the present disclosure can be used as a pharmaceutical composition for treating cancer. For example, a pharmaceutical composition inhibiting cancer cell proliferation, a pharmaceutical composition for inhibiting cancer metastasis, a pharmaceutical composition for inhibiting cancer recurrence, or a pharmaceutical composition for triggering a tumor immune response. Preferably, the pharmaceutical composition can be a tumor vaccine.

The kit for treating cancer of the present disclosure includes the immunothermosensitive composition of the present disclosure and a light supply device. The immunothermosensitive composition can absorb light energy generated by the light supply device to generate a mild thermal energy and maintain the mild thermal energy. Therefore, the immunothermosensitive composition has the dual effects of hyperthermia therapy and immunotherapy, thereby producing synergistic therapeutic effects, and greatly improving the cancer treatment effect. In addition, the kit for treating cancer of the present disclosure can modulate the tumor microenvironment by attracting the accumulation of CD3⁺ T cells and secreting granzyme B. Therefore, the kit for treating cancer of the present disclosure can induce the anti-tumor durable immunological memory in the treated individual against tumor metastasis or recurrence.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1. An immunothermosensitive composition, comprising:
a carrier formed by self-assembly of a hydrophilic amine-containing polymer and a conductive polymer to form a hydrophilic region and a hydrophobic region, wherein the hydrophilic region is located outside the hydrophobic region, and the conductive polymer has a covalent π bond; and
an immune adjuvant coated in the hydrophobic region of the carrier, wherein the immune adjuvant specifically binds to Toll-Like Receptor 7 (TLR7) and/or Toll-Like Receptor 8 (TLR8);
wherein the immunothermosensitive composition absorbs light energy to generate thermal energy and maintains a temperature greater than or equal to 39° C. and less than or equal to 45° C.

2. The immunothermosensitive composition of claim 1, wherein the hydrophilic amine-containing polymer is glycol chitosan (GCS), gelatin, O-carboxymethyl chitosan (CMCS), albumin, poly-L-lysine (PLL) or polyetherimide (PEI).

3. The immunothermosensitive composition of claim 1, wherein the conductive polymer is polyaniline (PANI), trans-polyacetylene (trans-PA), poly-p-phenylene (PPP), poly(p-phenylene vinylene) (PPV), poly(p-phenylene sulfide) (PPS), polypyrrole (PPy), polythiophene (PTh), poly(3,4-ethylenedioxythiophene) (PEDOT) or poly(9,9-di-n-octyl-2,7-fluorene) (PFO).

4. The immunothermosensitive composition of claim 1, wherein the immune adjuvant is resiquimod, imiquimod, gardiquimod, CL097, 3M-003, CL075, TL8-506 or motolimod.

5. The immunothermosensitive composition of claim 1, wherein per milligram of the carrier comprises 10 μg to 50 μg of the conductive polymer.

6. The immunothermosensitive composition of claim 1, wherein a surface charge of the immunothermosensitive composition ranges from 10 mV to 50 mV.

7. The immunothermosensitive composition of claim 1, wherein based on the immunothermographic composition is 100 parts by weight, a weight ratio of the carrier to the immune adjuvant is 80 parts by weight: 20 parts by weight to 95 parts by weight: 5 parts by weight.

8. A kit for treating cancer, comprising:
the immunothermosensitive composition of claim 1; and
a light supply device for irradiating the immunothermosensitive composition.

9. The kit for treating cancer of claim 8, wherein a light source of the light supply device is ultraviolet light (UV), near infrared light (NIR), far infrared light (FIR) or visible light (VIS).

10. A method for treating cancer comprising administering an effective amount of the immunothermosensitive composition of claim 1 to a subject in need for a treatment of cancer.

11. The method for treating cancer of claim 10, wherein the immunothermosensitive composition inhibits a cancer cell proliferation.

12. The method for treating cancer of claim 10, wherein the immunothermosensitive composition inhibits a cancer metastasis.

13. The method for treating cancer of claim 10, wherein the immunothermosensitive composition inhibits a cancer recurrence.

14. The method for treating cancer of claim 10, wherein the immunothermosensitive composition triggers a tumor immune response.

15. The method for treating cancer of claim 10, wherein the immunothermosensitive composition is used as a tumor vaccine.
